(19) United States
(12) Patent Application Publication
(10) Pub. No.: US 2008/0305514 A1
(43) Pub. Date: Dec. 11, 2008

(54) METHOD FOR DETECTING MICROBES

(75) Inventors: Kathleen G. Alford, Fort Worth, TX (US); Sheryll H. Handley, Fort Worth, TX (US); Barry A. Schlech, Burleson, TX (US); S. Paul Shannon, Arlington, TX (US); Ronald L. Smith, Arlington, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/106,887

(22) Filed: Apr. 21, 2008

Related U.S. Application Data
(60) Provisional application No. 60/942,308, filed on Jun. 6, 2007.

(51) Int. Cl. C12Q 1/04

(52) U.S. Cl. 435/34

(57) ABSTRACT

The present invention relates to methods for detecting microbes in a sample comprising filtering the sample through a fluid-permeable surface, contacting the surface with a viability stain, scanning the surface for viability stain to form a first scan, contacting the surface with a nucleic acid stain, scanning the surface for nucleic acid stain to form a second scan, and comparing said first scan and said second scan.

### CROSS-REFERENCE TO RELATED APPLICATION

This application claims priority under 35 U.S.C. §119 to U.S. Provisional Patent Application No. 60/942308, filed Jun. 6, 2007, the entire contents of which are incorporated herein by reference.

### TECHNICAL FIELD OF THE INVENTION

The present invention relates generally to methods of testing samples for the presence of microbes. The present invention further relates to methods for reducing false positive results when testing samples for microbes.

### BACKGROUND OF THE INVENTION

Procedures for detecting the presence of microbes such as bacteria and fungi in samples are used in a vast number of applications in a variety of fields. Water samples are tested to detect the presence of coliform bacteria, the presence of which can indicate that the samples may be contaminated by fecal matter and are unfit as a potable water source. Consumables made by food manufacturers are tested to ensure that undesirable microbes are not present. Many pharmaceutical companies and medical device manufacturers have product lines that must be devoid of viable microbes and are sampled to ensure the sterility of the finished product. Certain products (such those manufactured by the beer and wine industries) are tested using procedures for enumerating desirable microbes in samples.

While the standard plate count and direct microscopic count methods (Mesa et al. 2003, Biegala et al. 2002, Shopov et al. 2000, Hoff 1993) are the most commonly used methods to enumerate microbial cells (see review by Manafi et al. 1991), both suffer from quantitative and qualitative limitations. Microscopic techniques are labor intensive, highly variable, and unable to discriminate between living and dead microorganisms without chemical processing (McFeters et al. 1995, Kepner and Pratt 1994). Methods that rely on conventional culture techniques are limited by the time required for organisms to achieve density sufficient for detection. Moreover, culture-based methods are unable to enumerate organisms that are viable, but not cultureable (VBNC), or organisms with nutritional requirements not satisfied by the culture medium.

Pharmaceutical companies that manufacture sterile products have attempted to develop alternate technologies that are able to circumvent the inherent limitations of growth based assays. Compendial methods for ascertaining sterility of a solution dictate a minimum incubation period of fourteen days and do little to address organisms that are viable but non-cultureable. Solid-phase cytometric assays are viability-based techniques that have been evaluated as tools to enable the very rapid detection of microorganisms in many different products and as possible alternatives to growth-based sterility test methods (Lisle et al. 2004, Lemarchand et al. 2001, Jones et al. 1999). These techniques have been used to determine total viable counts in water and can determine the presence of specific microorganisms when used in conjunction with taxonomic probes (Rushton et al. 2000, Pyle et al. 1999) or monoclonal antibodies (Aurell et al. 2004).

One such assay system is the ChemScan® RDI (or Scan RDI™) microbial detection system (Chemunex, France), which employs a combination of direct fluorescent labeling techniques and solid phase laser scanning cytometry to rapidly enumerate viable microorganisms without the need for growth and multiplication (Mignon-Godefroy et al. 1997). The system has sufficient sensitivity to detect a single viable microorganism within 3 hours, without the need for growth and multiplication. Cells are collected from aqueous samples by filtration onto the surface of polyester membranes and treated with a proprietary combination of background and viability stains. The viability stain consists of a non-fluorescent membrane permeant substrate, similar to fluorescein diacetate, cleaved by non-specific esterases into a membrane impermeant chromophore. Cells with intact membranes accumulate the chromophore in the cytoplasm while those with compromised membranes are unable to retain the fluorescent probe (Breeuwer et al. 1995). Fluorescent events are recorded by the system and processed through a battery of discrimination parameters designed to differentiate labeled organisms from background noise and autofluorescing particulates. Identified events may then be validated as true positives using direct microscopic examination.

Despite the use of stringent discrimination parameters, a significant number of autofluorescing particulates with physical characteristics similar to microbial cells are often included in the event dataset for validation. While the viability staining protocol is considered non-destructive, in that cell morphology is not significantly altered, the long-term viability of a processed microorganism is profoundly affected. Efforts to confirm the biological nature of these fluorescent events by subsequent culture have been largely unsuccessful, thus impeding attempts to investigate the source and identity of contaminating microorganisms and increasing the probability of incurring the consequences of false positive results. Thus, improved methods for testing samples for the presence of microbes are desirable, particularly those methods that reduce the likelihood of generating false positive results.

### BRIEF SUMMARY OF THE INVENTION

One aspect of the present invention is a method for detecting microbes in a sample. The method comprises: (1) filtering the sample through a fluid-permeable surface; (2) contacting the surface with a viability stain; (3) scanning the surface for viability stain to form a first scan; (4) contacting the surface with a nucleic acid stain; (5) scanning the surface for nucleic acid stain to form a second scan; and (6) comparing the first scan and the second scan.

A second aspect of the present invention is a method for identifying false positive results associated with testing a sample for sterility comprising: (1) filtering said sample through a fluid-permeable surface; (2) contacting said surface with a fluorescent viability stain; (3) scanning said surface for fluorescent matter to form a first scan, said first scan comprising fluorescent matter position information; (4) contacting the fluid-permeable surface with a nucleic acid stain; (5) scanning the surface for nucleic acid stain to form a second scan, said second scan comprising nucleic acid stain position information; (6) comparing said first scan and said second scan position information; and (7) identifying at least one false positive if said first scan fluorescent matter position information does not match said second scan nucleic acid stain position information.

Yet another aspect of the present invention is a kit for reducing false positive results associated with a method for testing the sterility of a sample. Such a kit comprises a post-scan nucleic acid stain for detecting viable microbes and instructions for the use of the nucleic acid stain.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### BRIEF DESCRIPTION OF THE DRAWING

A more complete understanding of the present invention and the advantages thereof may be acquired by referring to the following description, taken in conjunction with the accompanying drawing in which like reference numbers indicate like features and wherein:

FIG. 1 shows the average biological staining efficiency (BSE) of microbial test strains relative to a 70% threshold; and

FIG. 2 shows representative photos of stained samples of vegetative microorganisms.

### DETAILED DESCRIPTION OF THE INVENTION

Embodiments of the present invention improve provide improved methods for detecting microbes using light scanning systems and decrease the likelihood of a generating false positive results. Embodiments of the present invention may be used to detect a variety of microbes, including without limitation, bacteria, viruses, yeast, fungi, spores, protozoa, parasites, etc.

Such embodiments use a nucleic acid stain to label nucleic acids in-situ and enable a user to confirm the biological nature of an “event”, or possible microbe, detected by light scanning. Amorphous particles and crystals and other non-viable matter do not react with nucleic acid stains. Thus, scan events from light scanning can be treated with nucleic acid stains and confirmed as a positive event or discounted as an artifact or non-viable event. Embodiments of the present invention were validated against six test reference stains and exceeded a biological staining efficiency threshold of 70%. The techniques of the embodiments can be used in combination with protocols that comprise the following steps: (1) filtering a sample through a fluid-permeable surface; (2) contacting the surface with a viability stain; and (3) scanning the surface for viability stain.

Fluid-permeable surfaces that may be used with embodiments of the present invention preferably are polymer membrane filters known to those of skill in the art. Such filters include, but are not limited to, polyester, cellulose, and nitrocellulose filters. Many other fluid-permeable surfaces are also known to those of skill in the art and may comprise, for example, ceramics, nylon, and hydrophobic materials. Such fluid-permeable surfaces must be amenable to scanning using coherent, non-coherent, visible, ultraviolet, and/or infrared light.

The viability stains preferably used with embodiments of the present invention include, but are not limited to, esterase substrate dyes. However, other embodiments of the present invention may use such known dyes as fluorescein. Fluorescein diffuses readily across membranes resulting in the loss of fluorescence intensity from active cells and an increase in non-specific staining of dead cells and non-cellular particles. Therefore, the esterase substrate dyes that have high intracellular retention were developed (Haugland, 1996, herein incorporated by reference in its entirety). Using these esterase substrate dyes, live cells are detected by a combination of functional internal enzyme and intact membrane. The reliance of the method on these two viable cell parameters increases the confidence of this approach. Moreover the dependence on enzyme activity for fluorescence means that these dyes are less prone to non-specific binding and fluorescence.

Samples usable with embodiments of the present invention include both liquid and gaseous fluids as well as soluble solids such as powders, tablets, suspensions, etc. Pharmaceutical compounds are particularly preferred for use as samples with embodiments of the present invention.

The nucleic acid stain used in preferred embodiments of the present invention is 4′,6-diamidino-2-phenylindole in isopropyl alcohol (Invitrogen Corporation, Carlsbad, Calif.). Invitrogen offers a series of nucleic acid stains that are permeant to most cells, although the rate of uptake and staining pattern may be cell dependent. Because the membrane of intact cells offers a barrier to entry of higher-affinity nucleic acid stains, a common practice has been to combine dyes to give the researcher the tools to more precisely understand the system being studied. The SYTO 13 green-fluorescent nucleic acid stain has been used in combination with ethidium bromide for studies of tissue cryopreservation (Lebaron et al. 1998), hexidium iodide for simultaneous viability and gram sign of clinically relevant bacteria (Roth et al. 1997), ethidium homodimer-1 for quantitation of neurotoxicity (Vaahtovuo et al. 2005) and with propidium iodide to detect the effects of surfactants on Escherichia coli viability (Sgorbati et al. 1996). With SYTO-staining combinations, staining may be done using the multiple stains simultaneously or sequentially; however, in preferred embodiments, the stains are applied sequentially.

One current light scanning system for detecting microbes is the ChemScan® RDI (or Scan RDI™) microbial detection system. This system employs a combination of direct fluorescent labeling techniques and solid phase laser scanning cytometry to rapidly enumerate viable microorganisms residing on a fluid-permeable membrane filter. Microorganisms with intact cytoplasmic membranes accumulate the fluorescent chromophore used in the system, which enables the instrument system to differentiate them from background noise. Putative microorganisms are subsequently verified by direct microscopic examination. Such a detection system is described in greater detail in U.S. Pat. No. 5,663,057, “Process for Rapid and Ultrasensitive Detection and Counting of Microorganisms by Fluorescence,” the entire contents of which are herein incorporated by reference. Despite the use of stringent discrimination parameters, a significant number of autofluorescing particulates with physical characteristics similar to microbial cells are often included in the validation dataset generated by this system and by other systems for detecting microbes. Such autofluorescing particulates can generate false positive results; false positive results are events or data that indicate the presence of a microbe when, in fact, no microbe is present. Embodiments of the present invention are preferably used in conjunction with the Scan RDT™ detection system.

### EXAMPLES

The following examples are presented to further illustrate selected embodiments of the present invention. When evaluating samples for events such as would occur in sterility testing, it is important to have a secondary tool to evaluate whether or not an event is actually a biological cell. Analysts use their training and experience to determine if the event has a characteristic shape of a cell or if it is a particle. A secondary staining technique was developed and validated against six sterility test reference strains for determining if an event is a microorganism or a particle.

### Microorganisms

Staphylococcus aureus ATCC 6538 and Pseudomonas aeruginosa ATCC 9027 were maintained on Soybean Casein-Digest Agar. Candida albicans ATCC 10231 was maintained on Sabouraud Dextrose Agar. Bacillus subtilis ATCC 6633, Aspergillus niger ATCC 16404 and Clostridium sporogenes ATCC 11437 were maintained as spore suspensions.

### Solid Phase Laser Cytometry

The Chemunex Scan RDI™ system consists of a laser-scanning unit equipped with a 488-nm argon laser and two photomultiplier tubes, with wavelength windows set for the green (500-530 nm) and amber (540-585 nm) regions of the emission spectrum of fluorescein. The signals produced are processed by a computer using a series of software discriminants that enable the instrument to differentiate between valid signals (labeled cells) and background noise (electronic interference or autofluorescent particles). Scan results are displayed as green spots on a computer generated scan map image of the membrane filter. An epifluorescence microscope (Olympus BX51), equipped with multiple filter sets (UV, FITC, TXRED, TRITC) and a motorized-stage driven by the laser scanning software, was used to confirm that the fluorescent events were viable biological cells.

### Validation Studies

Chemunex Fluorassure Integral Filtration Units (FIFU) were used to prepare replicate sample filters for each test organism. The results from three replicates were used to validate the method for each organism. 100 μL of each organism suspension containing between 10-200 organisms was placed in the FIFU unit and filtered under vacuum. After inoculating the filter, 1.0 mL of the CSE/CSM background stain (Chemunex) was added directly to the filter and vacuum filtered. The bottom portion of the FIFU was removed and attached to a labeling pad support whose pad was soaked with A16 (Chemunex). The filter on labeling pad support was placed in the incubator (30 to 35° C.) for one to three hours. Following incubation, the filter was transferred to a fresh labeling pad support whose pad was saturated with approximately 0.5 mL of prepared V6 solution (Chemunex). The filter on support was incubated at 30 to 35° C. for 30-45 minutes. Following the incubation on V6, the filter unit was placed onto a pre-wetted support pad situated on a scan membrane holder, placed into the ScanRDI reader and scanned by the system. After completion of the scan, the scan membrane holder was placed onto the motor driven stage of a custom fitted fluorescence microscope and the cells were visually confirmed under the FITC filter set. After validation of the events the scan was saved. The filter was aseptically removed from the scan membrane holder, placed back into the FIFU membrane carrier and attached to a sterile labeling pad support. 0.8 mL of nucleic acid stain (4′,6-diamidino-2-phenylindole in isopropyl alcohol) was added to the labeling pad. The filter on support was then incubated at room temperature, in the dark, for 60 to 90 minutes. Following incubation, the filter was placed onto a pre-wetted support pad sitting on a scan membrane holder. The scan membrane holder was placed onto the motorized stage of the microscope. The original scan map was called up and the computer drove the stage to each validated event. Under the UV filter set, each event site previously validated as a microbial cell was examined. The event was confirmed as biological if the cell fluoresced blue from the nucleic acid stain. The number of the validated events recorded from both the initial scan and staining regimen were used to calculate a biological staining efficiency (BSE) for each organism according to the formula: BSE=Count of Nucleic Acid Stained Cells/Count of Original Viable Cells.

### Results

TABLE 1

| | Count of PSSR Events/ | | |
|---|---|---|---|
| | Count of Initial Events | | |
| | (Biological Staining | | |
| Test Microorganism | Efficiency-BSE) (E) | | |
| Bacillus subtilis | 58/76 | 53/68 | 78/80 |
| ATCC 6633 | (76) | (78) | (98) |
| Candida albicans | 90/90 | 91/95 | 96/99 |
| ATCC 10231 | (100) | (96) | (97) |
| Clostridium sporogenes | 41/42 | 23/23 | 36/41 |
| ATCC 11437 | (98) | (100) | (88) |
| Staphylococcus aureus | 27/30 | 72/87 | 30/39 |
| ATCC 6538 | (90) | (83) | (77) |
| Pseudomonas aeruginosa | 13/17 | 35/37 | 23/30 |
| ATCC 9027 | (76) | (95) | (77) |
| Aspergillus niger | 162/163 | 193/205 | 95/102 |
| ATCC 16404 | (99) | (94) | (93) |

Table 1 shows the biological staining efficiency (BSE) for the nucleic acid stain (4′,6-diamidino-2-phenylindole in isopropyl alcohol) tested using 10-200 cells of pure cultures of Staphylococcus aureus, Pseudomonas aeruginosa, Candida albicans, Bacillus subtilis, Aspergillus niger and Clostridium sporogenes. BSE is the percentage of initial events seen after the viability stain that were also observed using the nucleic acid stain (4′,6-diamidino-2-phenylindole in isopropyl alcohol). Based on the results of these validation tests, all compendial organisms exceeded a Biological Staining Efficiency (BSE) threshold of 70%.

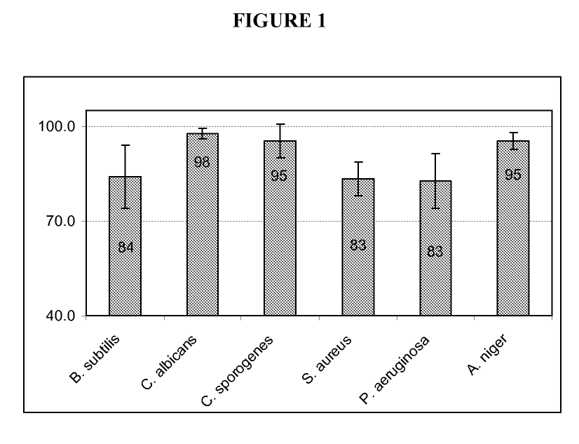

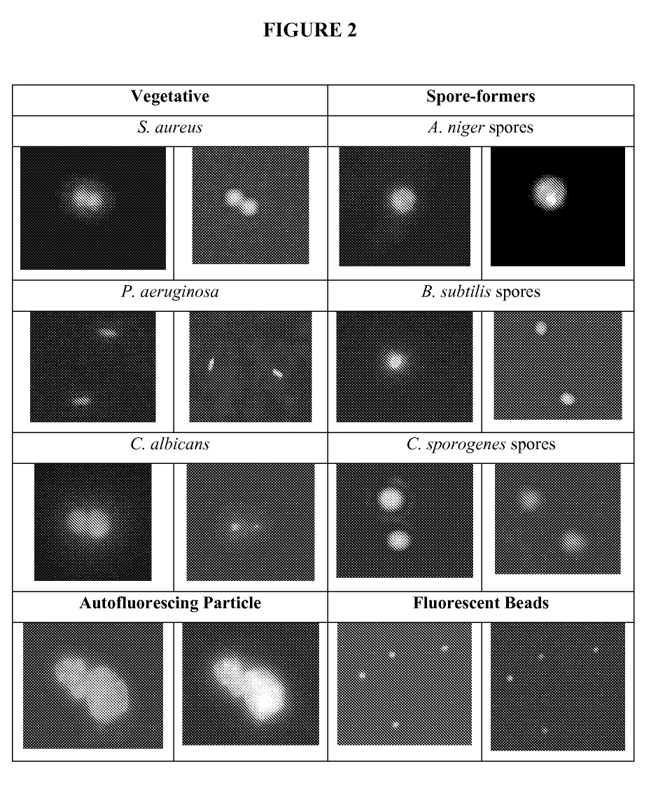

The non-sporeforming strains showed a BSE between 83 and 97%. The staining efficiency appeared to be based on cell size as both S. aureus and P. aeruginosa had a BSE of approximately 83% while the larger C. albicans had the highest at 97%. The spore-formers displayed a similar profile with B. subtilis staining at 84% and the larger spores of A. niger and C. sporogenes staining at 95%. All organisms exceeded the 70% BSE threshold, as shown in FIG. 1. Photographic images from all organisms as well as inert particles are displayed in FIG. 2. Representative samples of vegetative microorganisms (S. aureus, P. aeruginosa, C. albicans) and spores (A. niger, B. subtilis, C. sporogenes) stained with viability stain fluorescein (viewed under FITC filter) appear as green, and nucleic acid stain (viewed under UV filter set) appear as blue. Unstained autofluorescing particle and fluorescent beads are viewed under both FITC (green) and UV (blue) filter sets.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

Aurell et al., “Rapid detection and enumeration of Legionella pneumophila in hot water systems by solid-phase cytometry”, Applied and Environmental Microbiology, Vol. 70:1651-1657, 2004.

Biegala et al., “Identification of bacteria associated with dinoflagellates (Dinophyceae) Alexandrium spp. using tyramide signal amplification-fluorescent in situ hybridization and confocal microscopy”, Journal Phycol., Vol. 38:404-411, 2002.

Breeuwer et al., “Characterization of uptake and hydrolysis of fluorescein diacetate and carboxyfluorescein diacetate by intracellular esterases in Saccharomyces cerevisiae, which result in accumulation of fluorescent product”, Applied and Environmental Microbiology, Vol. 61:1614-1619, 1995.

Haugland, R., Handbook of fluorescent probes and research chemicals. Molecular Probes Inc., Eugene, Oreg. 1996

Hoff, K. A., “Total and specific bacterial counts by simultaneous staining with DAPI and fluorochrome-labeled antibodies”, In: P. F. Kemp, B. F. Sherr, E. B. Sherr and J. J. Cole, Editors, Handbook of Methods in Aquatic Microbial Ecology, Lewis Publishers, Boca Raton, Fla., USA, pp. 149-154, 1993.

Jones et al., “Solid-phase, laser-scanning cytometry: a new two-hour method for the enumeration of microorganisms in Pharmaceutical water”, Pharmacopeial Forum, Vol. 25:7626-7645, 1999.

Kepner et al., “Use of fluorochromes for direct enumeration of total bacteria in environmental samples: past and present”, Microbiological Reviews, Vol. 58:603-615, 1994.

Lebaron et al., “Effectiveness of SYTOX green stain for bacterial viability assessment,” Applied and Environmental Microbiology, Vol. 64:2697-2700, 1998.

Lemarchand et al., “Comparative assessment of epifluorescence microscopy, flow cytometry and solid-phase cytometry used in enumeration of specific bacteria in water”, Aquatic Microbial Ecology, Vol. 25:301-309, 2001.

Lisle et al., “Comparison of fluorescence microscopy and solid-phase cytometry methods for counting bacteria in water”, Applied and Environmental Microbiology, Vol. 70:5343-5348, 2004.

Manafi et al., “Fluorogenic and chromogenic substrates used in bacterial diagnostics”, Microbiological Reviews, Vol. 55:335-348, 1991.

McFeters et al., “Physiological assessment of bacteria using fluorochromes”, Journal of Microbiological Methods, Vol. 21:1-13, 1995.

Mesa et al., “Use of the Direct Epifluorescent Filter Technique for the Enumeration of Viable and Total Acetic Acid Bacteria from Vinegar Fermentation”, Journal of Fluorescence, Vol. 13:261-265, 2003.

Mignon-Godefroy et al., “Solid phase cytometry for detection of rare events”, Cytometry, Vol. 27:336-344, 1997.

Pyle et al., “Sensitive detection of Escherichia coli O157:H7 in food and water by immunomagnetic separation and solid-phase laser cytometry”, Applied and Environmental Microbiology, Vol. 65:1966-1972, 1999.

Roth et al., “Bacterial viability and antibiotic susceptibility testing with SYTOX green nucleic acid stain”, Applied and Environmental Microbiology, Vol. 63:2421-2431, 1997.

Rushton et al., “An evaluation of a laser scanning device for the detection of Cryptosporidium parvum in treated water samples”, Letters in Applied Microbiology, Vol. 30:303-307, 2000.

Sgorbati et al., “Characterization of number, DNA content, viability and cell size of bacteria from natural environments using DAPI PI dual staining and flow cytometry”, Minerva Biotecnologica, Vol. 8:9-15, 1996.

Shopov et al., “Improvements in image analysis and fluorescence microscopy to discriminate and enumerate bacteria and viruses in aquatic samples”, Aquatic Microbial Ecology, Vol. 22:103-110, 2000.

J. Vaahtovuo et al., “Quantification of bacteria in human feces using 16S rRNA-hybridization, DNA-staining and flow cytometry”, Journal Microbiological Methods, Vol. 63:276-286, 2005.

What is claimed is:

1. A method for detecting microbes in a sample comprising:
filtering said sample through a fluid-permeable surface;
contacting said surface with a viability stain;
scanning said surface for viability stain to form a first scan;
contacting said surface with a nucleic acid stain;
scanning the surface for nucleic acid stain to form a second scan; and
comparing said first scan and said second scan.

2. The method of claim 1 wherein said first scan and said second scan are each a set of position coordinates.

3. The method of claim 2 wherein said first scan identifies viability stain position coordinates and wherein said second scan identifies nucleic acid stain position coordinates.

4. The method of claim 3 wherein said comparing identifies a microbe if said first scan and said second scan have at least one common position coordinate.

5. The method of claim 3 wherein said comparing identifies a false positive if a first scan position coordinate is not present in said second scan.

6. The method of claim 1 wherein said viability stain is an esterase substrate dye.

7. The method of claim 1 wherein said nucleic acid stain is:
4′,6-diamidino-2-phenylindole in isopropyl alcohol.

8. The method of claim 1 wherein said scanning comprises scanning with light selected from the group consisting of: coherent, non-coherent, visible, ultraviolet, infrared, and combinations thereof.

9. The method of claim 1 wherein said scanning comprises scanning using a microscope.

10. A method for identifying false positive results associated with testing a sample for sterility comprising:
filtering said sample through a fluid-permeable surface;
contacting said surface with a fluorescent viability stain;
scanning said surface for fluorescent matter to form a first scan, said first scan comprising fluorescent matter position information;
contacting the fluid-permeable surface with a nucleic acid stain;
scanning the surface for nucleic acid stain to form a second scan, said second scan comprising nucleic acid stain position information;
comparing said first scan and said second scan position information; and
identifying at least one false positive if said first scan fluorescent matter position information does not match said second scan nucleic acid stain position information.

11. The method of claim 10 wherein said first scan position information and said second scan position information are each a set of position coordinates.

12. The method of claim 10 wherein said viability stain is:
an esterase substrate dye.

13. The method of claim 10 wherein said nucleic acid stain is:
4′,6-diamidino-2-phenylindole in isopropyl alcohol.

14. The method of claim 10 wherein said scanning comprises scanning with light selected from the group consisting of:
coherent, non-coherent, visible, ultraviolet, infrared, and combinations thereof.

15. The method of claim 10 wherein said scanning comprises scanning using a microscope.

16. A kit for reducing false positive results associated with a method for testing the sterility of a sample comprising:
a post-scan nucleic acid stain for detecting viable microbes; and
instructions for the use thereof.

17. The kit of claim 16 wherein said stain is:
4′,6-diamidino-2-phenylindole in isopropyl alcohol.
